(19) United States
(12) Patent Application Publication
(10) Pub. No.: US 2022/0411743 A1
(43) Pub. Date: Dec. 29, 2022

(54) NEW PROCESS OF EXTRACTING PROTEIN

(71) Applicant: Affibody AB, Solna (SE)

(72) Inventors: Finn Dunås, Bromma (SE); Belinda Röjstäter, Solna (SE); Tomas Moks, Täby (SE)

(21) Appl. No.: 17/772,808

(22) PCT Filed: Nov. 9, 2020
(86) PCT No.: PCT/EP2020/081420

§ 371 Date: Apr. 28, 2022

(30) Foreign Application Priority Data
Nov. 8, 2019 (EP) 19208153.7

(51) Int. Cl. C12M 1/00; C12N 1/06; C07K 1/14

(52) U.S. Cl. CPC C12M 47/06; C12N 1/06; C07K 1/145

(57) ABSTRACT

A method of extracting a cytoplasmic or periplasmic protein. The method comprises provision of a first cell suspension comprising cells, the cells containing a cytoplasmic or periplasmic protein of interest to be extracted, the first cell suspension having a first temperature, and heating of the first cell suspension to an operating temperature, at which operating temperature at least a fraction of the cells is subject to heat-induced lysis and at least a fraction of the cytoplasmic or periplasmic protein of interest to be extracted is not subject to irreversible denaturation. The heating of the first cell suspension comprises provision of an aqueous solution, the aqueous solution having a second temperature that is higher than the first temperature, and mixing of the first cell suspension with the aqueous solution, thereby obtaining a second cell suspension, the second cell suspension having a third temperature that is higher than the first temperature. A system for extracting a cytoplasmic or periplasmic protein. The system comprises, i.a., a static mixer.

### TECHNICAL FIELD

The present application relates to a method of extracting a cytoplasmic or periplasmic protein, the method comprising heating of a cell suspension to a temperature, at which temperature heat-induced lysis of cells containing a cytoplasmic or periplasmic protein of interest to be extracted occurs and irreversible denaturation of the cytoplasmic or periplasmic protein of interest to be extracted does not occur. The present application also relates to a system for extracting a cytoplasmic or periplasmic protein.

### BACKGROUND ART

Large-scale manufacturing of recombinant proteins for medical and biotechnological applications requires process development and optimization to meet the demands of a cost-efficient and reproducible production process yielding a high-quality end product. Processes that work well in small-scale production may not be feasible for large-scale production, for technical or economic reasons. Process development in conditions mimicking potential large-scale processes, followed by scale up of the developed production process, are essential if industrial manufacture is the target.

In the production of recombinant proteins expressed by cells, efficient cell lysis and recovery of the desired product is of crucial importance. Several methods for lysing cells have been developed and described. These include mechanical homogenization, ultrasonic homogenization, pressure homogenization, heat treatment, freeze/thaw cycles as well as osmotic and chemical lysis. The method of choice will depend on various factors such as the properties of the protein of interest that is to be extracted from the cells, the subcellular localization of said protein, the volumes involved and the required throughput.

For extraction of thermostable proteins, lysis by heat treatment can be advantageous as it may result in precipitation of unwanted host cell proteins and/or removal of insoluble aggregates and hence aid the subsequent purification. Furthermore, when used at a screening stage lysis by heat treatment will favour thermostable variants that are more likely to result in larger yields of correctly folded proteins.

However, for large-scale production, the time required to heat and cool the cell suspension could affect the overall yield as the protein of interest may start to precipitate or degrade. The time required to heat and cool the cell suspension could additionally cause problems with generation of product related impurities. Thus, lysis by heat treatment is not optimal for large-scale production of proteins by cell expression as heating and cooling in larger bioreactors or vessels takes long time and thereby the result of the extraction process is poorly controlled. Hence, there is a need for improved extraction procedures to improve the process efficiency and the yield of proteins in large-scale cell expression processes.

### SUMMARY OF THE INVENTION

It is an object of the present invention to provide efficient extraction of a cytoplasmic or periplasmic protein of interest, i.e. to provide a high yield and/or quality of such protein, particularly in large-scale production. It is another object of the present invention to provide extraction of a cytoplasmic or periplasmic protein of interest while avoiding or reducing precipitation or degradation of the protein of interest to be extracted. It is a further object of the present invention to provide extraction of a cytoplasmic or periplasmic protein of interest while avoiding or reducing generation of product related impurities.

These objects as well as other objects of the invention, which should be apparent to a person skilled in the art after having studied the description below, are, in one aspect of the invention, accomplished by a method of extracting a cytoplasmic or periplasmic protein, the method comprising:
provision of a first cell suspension comprising cells, the cells containing a cytoplasmic or periplasmic protein of interest to be extracted, the first cell suspension having a first temperature, and
heating of the first cell suspension to an operating temperature, at which operating temperature at least a fraction of the cells is subject to heat-induced lysis and at least a fraction of the cytoplasmic or periplasmic protein of interest to be extracted is not subject to irreversible denaturation,
   wherein the heating of the first cell suspension comprises:
provision of an aqueous solution, the aqueous solution having a second temperature that is higher than the first temperature, and
mixing of the first cell suspension with the aqueous solution, thereby obtaining a second cell suspension, the second cell suspension having a third temperature that is higher than the first temperature.

The method of extracting a cytoplasmic or periplasmic protein may thus, in other words, comprise:
provision of a first cell suspension comprising cells, the cells containing a cytoplasmic or periplasmic protein of interest to be extracted, the first cell suspension having a first temperature, and
heating of the first cell suspension to an operating temperature, at which operating temperature heat-induced lysis of the cells occurs and irreversible denaturation of the cytoplasmic or periplasmic protein of interest to be extracted does not occur,
   wherein the heating of the first cell suspension comprises:
provision of an aqueous solution, the aqueous solution having a second temperature that is higher than the first temperature, and
mixing of the first cell suspension with the aqueous solution, thereby obtaining a second cell suspension, the second cell suspension having a third temperature that is higher than the first temperature.

The heating of the first cell suspension to the operating temperature thus results in release of the cytoplasmic or periplasmic protein of interest, thereby providing released cytoplasmic or periplasmic protein of interest. Mixing of the first cell suspension, comprising the cells to be lysed, with a warmer solution allows for fast heating of the cell suspension towards the lysis temperature, allowing in turn for increased recovery of the protein of interest to be extracted. The first cell suspension may be mixed with the aqueous solution in batch mode or continuously, preferably continuously. The mixing ratio between the first cell suspension and the aqueous solution may be in the range of 1:0.1 to 1:14, preferably in the range of 1:1 to 1:14, more preferably in the range of 1:2 to 1:12, more preferably in the range of 1:3 to 1:10. It is contemplated that the dilution achieved by mixing the first cell suspension with the aqueous solution reduces the viscosity and the risk that the protein of interest to be extracted adheres to, and/or co-precipitates with, other proteins.

With the method according to the present invention, the third temperature may be reached by subjecting the cells to heating for no more than 10 min, such in the range of 0.1 s to 10 min, preferably for no more than 5 min, such as in the range of 0.1 s to 5 min, more preferably for no more than 1 min, such as in the range of 0.1 s to 1 min, more preferably for no more than 10 s, such as in the range of 0.1 s to 10 s, most preferably for no more than 1 s, such as in the range of 0.1 s to 1 s.

Herein, the extraction of a cytoplasmic or periplasmic protein of interest refers to release of a protein of interest from the cytoplasm or periplasm of the cell where it was expressed. Herein, the operating temperature, at which heat-induced lysis of the cells occurs and at which irreversible denaturation of the cytoplasmic or periplasmic protein of interest to be extracted does not occur, relates to a temperature at which at least a minor fraction, preferably a major fraction or essentially all, of the cells are subject to lysis and at which at least a minor fraction, preferably a major fraction or essentially all, of the protein molecules of the cytoplasmic or periplasmic protein of interest to be extracted are not subject to irreversible denaturation. Herein, denaturation refers to the process in which proteins partially or totally lose the quaternary, tertiary and/or secondary structure that is present in their native state.

The aqueous solution is typically a buffer solution, as is commonly utilized in the processing of cells and proteins. In other words, the aqueous solution has a composition that is suitable for use in the present method. A person skilled in the art is thus able to adapt the composition of the aqueous solution in order to optimise the recovery of the protein of interest to be extracted. Adaptation of the composition of the aqueous solution typically involves selection of appropriate buffering components, of an appropriate salt concentration, conductivity and/or pH, and/or of any additives. Such additives may include those that protect the protein of interest against modification or degradation or that enhances the precipitation of unwanted cell components.

The aqueous solution may thus have a pH and/or a conductivity, and/or may contain an additive, that enhances the extraction of the protein of interest. Further, the aqueous solution may have a pH and/or a conductivity, and/or may contain an additive, that enhances the protection of the protein of interest against modification, degradation, misfolding or precipitation. Further, the aqueous solution may have a pH and/or a conductivity, and/or may contain an additive, that enhances the denaturation or precipitation of an unwanted cell component, such as host cell proteins, DNA, RNA, endotoxins or other cell components. Further, the aqueous solution may have a pH and/or a conductivity, and/or may contain an additive, that influences, preferably lowers, the temperature at which lysis of the cells occurs.

The first cell suspension is typically provided by subjecting a cell culture to centrifugation or filtration. It is also possible to provide the first cell suspension by mixing of frozen cell pellet, preferably obtained from a cell culture by centrifugation or filtration, with a warm buffer solution.

The first temperature, i.e. the temperature of the provided cell suspension, comprising the cells containing the protein of interest to be extracted, may be in the range of 0 to 37° C., preferably in the range of 2 to 37° C., more preferably in the range of 8 to 30° C., more preferably in the range of 18 to 25° C. The first temperature may alternatively be below 0° C., the cell suspension then comprising an anti-freeze formulation such as glycerol.

The operating temperature, i.e. the temperature at which lysis of the cells occur, may be below 90° C., such as in the range of 20 to 90° C., preferably in the range of 40 to 90° C., more preferably in the range of 50 to 90° C., more preferably in the range of 60 to 90° C., more preferably in the range of 70 to 90° C., more preferably in the range of 70 to 85° C., most preferably in the range of 75 to 85° C.

The second temperature, i.e. the temperature of the provided aqueous solution, may be below 110° C., such as in the range of 40 to 110° C., preferably in the range of 50 to 110° C., more preferably in the range of 60 to 99° C., more preferably in the range of 70 to 99° C., more preferably in the range of 80 to 99° C., more preferably in the range of 90 to 99° C., most preferably in the range of 90 to 95° C.

The third temperature, i.e. the temperature of the second cell suspension obtained by mixing of the first cell suspension and the aqueous solution, may be below 90° C., such as in the range of 40 to 90° C., preferably in the range of 50 to 90° C., more preferably in the range of 60 to 85° C., more preferably in the range of 65 to 85° C., more preferably in the range of 65 to 80° C., more preferably in the range of 65 to 78° C., most preferably in the range of 68 to 78° C. The third temperature may alternatively be in the in the range of 70 to 80° C. At the third temperature, at least a fraction of the cytoplasmic or periplasmic protein of interest to be extracted is preferably not subject to irreversible denaturation.

The heating of the first cell suspension may further comprise heating of the second cell suspension from the third temperature to the operating temperature. The heating of the first cell suspension by mixing with a warmer solution may thus be followed by additional heating towards the lysis temperature. Such additional heating is appropriate if the operating temperature cannot be reached merely by mixing of the first cell suspension with a warmer solution, which may be the case when limitations apply to the mixing ratio and the temperatures of the fluids to be mixed. Furthermore, such additional heating allows for accurate control of the temperature of the second cell suspension. The heating of the second cell suspension from the third temperature to the operating temperature is preferably performed by indirect heat exchange, such as in a tube heat exchanger or a plate heat exchanger.

It is preferred that the third temperature is no more than 10° C. lower, preferably no more than 5° C. lower, than the operating temperature. It is desirable to heat the first cell suspension towards the lysis temperature to large extent by mixing it with a warmer solution. It is thus advantageous if the third temperature is close to the operating temperature. A remaining temperature difference of 5 or 10° C. would allow for fine tuning of the temperature by means of additional heating.

Alternatively, the heating of the first cell suspension may further comprise cooling of the second cell suspension from the third temperature to the operating temperature. The heating of the first cell suspension by mixing with a warmer solution may thus be followed by cooling. Such cooling is appropriate if a temperature higher than the desirable operating temperature is reached by mixing of the first cell suspension with a warmer solution. Furthermore, such cooling allows for accurate control of the temperature of the second cell suspension. The cooling of the second cell suspension from the third temperature to the operating temperature is preferably performed by indirect heat exchange, such as in a tube heat exchanger or a plate heat exchanger. It is preferred that the third temperature is no more than 10° C. higher, preferably no more than 5° C. higher, than the operating temperature. As mentioned above, a remaining temperature difference of 5 or 10° C. would allow for fine tuning of the temperature by means of cooling.

Alternatively, the third temperature may be the operating temperature. In cases when the operating temperature may be directly reached, with appropriate precision, by mixing of the first cell suspension with a warmer solution, additional heating towards the lysis temperature may be dispensed with.

The method may further comprise maintenance of the second cell suspension at the operating temperature. Whereas keeping of the cell suspensions at increased temperature for prolonged periods, such as during heating and subsequent cooling of the suspensions, may negatively affect the recovery of the protein of interest to be extracted, it may not be sufficient for optimal extraction to merely reach the lysis temperature. It is thus advantageous to include in the extraction method maintenance, for a period of time, of the second cell suspension at the operating temperature. Herein, maintenance at the operating temperature refers to maintenance substantially at the operating temperature, i.e. also at such lower temperature that may be the consequence of any undesirable heat loss from the second cell suspension. The second cell suspension may be maintained at the operating temperature for a time period in the range of 1 s to 20 min or in the range of 10 s to 20 min, preferably in the range of 1 s to 10 min or in the range of 10 s to 10 min, more preferably in the range of 1 s to 5 min or in the range of 10 s to 5 min, most preferably in the range of 10 s to 4 min, such as in the range of 10 s to 30 s or in the range of 1 min to 4 min.

The method may further comprise cooling of the second cell suspension from the operating temperature to a fourth temperature, the fourth temperature preferably being a temperature at which at least a fraction of reversibly denatured cytoplasmic or periplasmic protein of interest to be extracted is subject to renaturation. The method may thus, in other words, further comprise cooling of the second cell suspension from the operating temperature to a fourth temperature, the fourth temperature preferably being a temperature at which renaturation of reversibly denatured cytoplasmic or periplasmic protein of interest to be extracted occurs. Maintaining the cell suspensions at increased temperature for prolonged periods may, as already mentioned, negatively affect the recovery of the protein of interest to be extracted. It is thus advantageous to include in the extraction method cooling of the second cell suspension from the operating temperature to a fourth temperature. For the same reason, it is advantageous to cool the second cell suspension before any subsequent procedure for separation of the protein of interest to be extracted from cell debris and/or native host cell proteins. In order to be able to finally recover the protein of interest to be extracted in its native form, it is advantageous that the fourth temperature is a temperature at which renaturation of reversibly denatured cytoplasmic or periplasmic protein of interest to be extracted occurs. Herein, this temperature, at which renaturation of reversibly denatured cytoplasmic or periplasmic protein of interest to be extracted occurs, relates to a temperature at which at least a minor fraction, preferably a major fraction or essentially all, of any reversibly denatured protein molecules of the protein of interest to be extracted is subject to renaturation to their native state. The fourth temperature may be in the range of 2 to 37° C., preferably in the range of 8 to 30° C. or in the range of 25 to 37° C., more preferably in the range of 18 to 25° C.

It is preferred that the residence time at a temperature that is higher than both the first temperature and the fourth temperature is no more than 20 min, such in the range of 1 s to 20 min or in the range of 10 s to 20 min, preferably no more than 10 min, such as in the range of 1 s to 10 min or in the range of 10 s to 10 min, more preferably no more than 5 min, such as in the range of 1 s to 5 min or in the range of 10 s to 5 min.

After extraction of the cytoplasmic or periplasmic protein of interest and possibly cooling of the cell suspension, the method may further comprise separation of the cytoplasmic or periplasmic protein of interest from cell debris and/or native host cell proteins. Such separation methods are well known to a person skilled in the art and typically comprise precipitation, filtration, centrifugation, and/or one or more forms of chromatography.

The cells may be prokaryotic cells, such as E. coli cells, or eukaryotic cells.

The cytoplasmic or periplasmic protein of interest to be extracted may comprise a three-helix bundle protein domain of a bacterial receptor protein, or a variant thereof. In particular embodiments, said three-helix bundle protein domain is selected from domains of bacterial receptor proteins. Non-limiting examples of such domains are i) the five different three-helical domains of Protein A from Staphylococcus aureus, such as domain B, and derivatives thereof. In some embodiments, the three-helical bundle protein domain is a variant of protein Z, which is derived from domain B of staphylococcal Protein A (Wahlberg E et al, 2003, PNAS 100(6):3185-3190), and ii) the albumin binding domain (ABD) of streptococcal Protein G (Kraulis et al, FEBS Lett 378:190, 1996) or a derivative thereof.

The mixing of the first cell suspension with the aqueous solution may be performed in a static mixer or in an agitated vessel, preferably in a static mixer. As is conventional in the art, the static mixer may be a pipe containing a series of stationary blades, typically helical blades, or a lattice of bars, typically intermeshing and/or interconnecting bars. It is preferred that said mixing is performed in continuous mode in a static mixer. When a flow of the first cell suspension meets a flow of the aqueous solution in a static mixer, the second cell suspension, having a higher temperature than the first cell suspension provided, is obtained virtually instantly.

The above-mentioned objects are, in another aspect of the invention, accomplished by a system for extracting a cytoplasmic or periplasmic protein of interest, the system comprising:
a cell suspension supply conduit having an inlet and an outlet, the inlet of the cell suspension supply conduit being connectable to a cell suspension container;
an aqueous solution supply conduit having an inlet and an outlet, the inlet of the aqueous solution supply conduit being connectable to an aqueous solution container;
a static mixer having at least one inlet and an outlet, the at least one inlet of the static mixer being in liquid communication with the outlet of the cell suspension supply conduit and with the outlet of the aqueous solution supply conduit;
a first heat exchanger having an inlet for cell suspension to be heated and an outlet for heated cell suspension, the inlet of the first heat exchanger being in liquid communication with the outlet of the static mixer;
a second heat exchanger having an inlet for cell suspension to be cooled and an outlet for cooled cell suspension, the inlet of the second heat exchanger being in liquid communication with the outlet of the first heat exchanger;
a discharge conduit having an inlet and an outlet, the inlet of the discharge conduit being in liquid communication with the outlet of the second heat exchanger and the outlet of the discharge conduit being connectable to a protein suspension container or to a protein suspension treatment system.

The system is suitable for performing the method disclosed above. The static mixer provides for fast heating of the cell suspension towards the lysis temperature, allowing in turn for increased recovery of the protein of interest to be extracted. As is conventional in the art, the static mixer may be a pipe containing a series of stationary blades, typically helical blades, or a lattice of bars, typically intermeshing and/or interconnecting bars. The static mixer may have one inlet, which is in liquid communication with both the outlet of the cell suspension supply conduit and the outlet of the aqueous solution supply conduit, or two inlets, one of which is in liquid communication with the outlet of the cell suspension supply conduit and the other of which is in liquid communication with the outlet of the aqueous solution supply conduit.

The first heat exchanger and the second heat exchanger may, independently, be a tube heat exchanger, a plate heat exchanger or a conduit being surrounded by a jacket or vessel.

The system may further comprise a holding conduit providing liquid communication between the outlet of the first heat exchanger and the inlet of the second heat exchanger, the holding conduit preferably being surrounded by a jacket or vessel, or by a heat insulating material, or by a heating blanket, such as an electrical heating blanket. The holding unit provides an opportunity to maintain, for a period of time, the temperature of the cell suspension substantially as achieved by the first heat exchanger. The residence time in the holding unit may be in the range of 1 s to 20 min or in the range of 10 s to 20 min, preferably in the range of 1 s to 10 min or in the range of 10 s to 10 min, more preferably in the range of 1 s to 5 min or in the range of 10 s to 5 min. As is common practice, a desired residence time may be obtained by selection of a suitable volume for the holding unit in relation to the flow rate of the cell suspension or vice versa.

The cell suspension supply conduit may comprise a pump for transporting a cell suspension towards the outlet of the cell suspension supply conduit. The aqueous solution supply conduit may comprise a pump for transporting an aqueous solution towards the outlet of the aqueous solution supply conduit. One or both of these pumps may be a positive displacement pump, such as a peristaltic pump.

The system may further comprise at least one heating unit providing heating medium to the first heat exchanger and/or to the jacket or vessel of the holding unit.

The system may be adapted to operate at the temperatures and/or flows disclosed elsewhere herein. It is preferred that the flow through the static mixer, the first heat exchanger, the holding unit and the second heat exchanger, as well as through the conduits connecting them, is turbulent, so as to reduce retention of, e.g., cell debris or proteins in pipes and equipment.

### BRIEF DESCRIPTION OF THE DRAWINGS

FIG. 1 is a schematic illustration of a system according to the present invention.

FIG. 2 shows the SDS-PAGE analysis of Example 3.

FIG. 3 shows the SDS-PAGE analysis of Example 4.

### DETAILED DESCRIPTION OF A PREFERRED EMBODIMENT

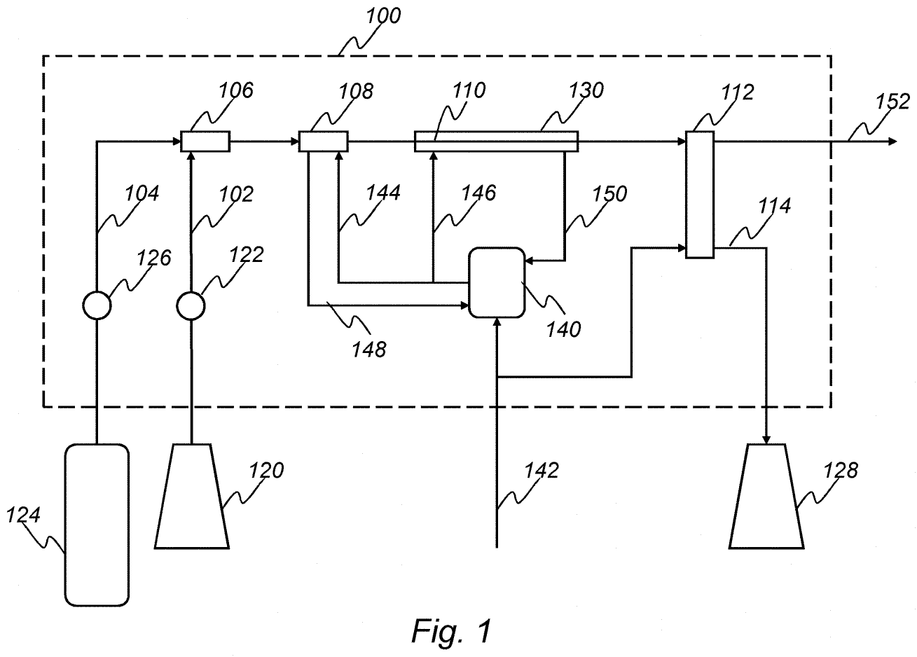

FIG. 1 shows a system 100 for extracting a cytoplasmic or periplasmic protein. The system 100 comprises a cell suspension supply conduit 102 and an aqueous solution supply conduit 104, which are both connected to a static mixer 106. The system 100 further comprises a first heat exchanger 108, a holding unit 110 and a second heat exchanger 112, which are connected in series. The static mixer 106 is connected to the first heat exchanger 108. The system further comprises a discharge conduit 114. The second heat exchanger 112 is connected to the discharge conduit 114.

The cell suspension supply conduit 102 is connected to a cell suspension container 120 and comprises a peristaltic pump 122. The aqueous solution supply conduit 104 is connected to an aqueous solution container 124 and comprises a peristaltic pump 126. The discharge conduit 114 is connected to protein suspension container 128. The holding unit 110 is provided with a jacket 130.

During operation of the system, the cell suspension container 120 provides a first cell suspension at room temperature whereas the aqueous solution container 124 provides a buffer solution at 95° C. The pumps 122, 126 transport the first cell suspension and the buffer solution from the containers 120, 124 to the static mixer 106, where the cell suspension and the aqueous solution are mixed in a ratio of 1:5, resulting in a second cell suspension at approx. 70° C. The second cell suspension is passed to the first heat exchanger 108, where the temperature of the second cell suspension is raised to 75° C. The second cell suspension is passed from the first heat exchanger 108, via the holding unit 110 to the second heat exchanger 112, where the temperature of the second cell suspension is lowered to 25° C. The second cell suspension has a residence time in the holding unit 110 of 5 min. The second cell suspension is passed from the second heat exchanger 112 via the discharge conduit 114 to the protein suspension container 128, from where it may be collected for further treatment.

The system 100 further comprises a heating unit 140. The heating unit is provided with tap water via a conduit 142. The heating unit 140 heats the tap water and provides the first heat exchanger 108 and the jacket 130 with heating medium via conduits 144 and 146, respectively. The heating medium is returned to the heating unit 130 via conduits 148 and 150, respectively. The tap water in conduit 142 also provides the second heat exchanger 112 with cooling medium. The cooling medium is discharged via a conduit 152.

### Examples

### Example 1, Heat-Induced Extraction of BPEP01

The description in this Example refers to cultivation, heat-induced extraction involving use of a static mixer, and subsequent analysis, of two repeated production batches of an approximately 19 kDa polypeptide, referred to as BPEP01, comprising two copies of a Z variant (Z01) and an albumin binding domain derived from GA3 of Streptococcal protein G. A comparison with heat treatment using a fermenter is included.

### Materials and Methods

Cultivation: The scale of the cultivations was either 6 L or 20 L. E. coli T7E2 cells (GeneBridges) were transformed with plasmids containing the gene fragment of the product. A Research Cell Bank (RCB) was generated using Vegitone LB-medium (Sigma-Aldrich) containing 50 mg/l kanamycin. When the culture had reached OD600=0.94, glycerol was added to a final concentration of 15%, and the culture was aliquoted into vials (1 ml/vial), which were frozen at −80° C.

Shake flask medium (6.7 g/l Yeast Nitrogen base (Becton Dickinson), 5.5 g/l glucose monohydrate, 7 g/l dipotassium monohydrogen phosphate, 1 g/l trisodium citrate dihydrate, 50 mg/ml kanamycin) was inoculated with 200 μl/l of a thawed RCB vial. After incubation at 30° C. to an OD600>4, a fermenter containing medium (3.75 g/l ammonium sulphate, 3.3 g/l dipotassium monohydrogen phosphate, 4.95 g/l monopotassium dihydrogen phosphate, 1.88 g/l trisodium citrate dihydrate, 1 ml/l antifoam 204 (Sigma-Aldrich), 6.1 mol/l magnesium sulphate, 50 mg/l kanamycin, 1.2 g/l glucose, 74 mg/l iron (III) chloride hexahydrate, 24 mg/l zinc sulfate heptahydrate, 4 mg/l copper (II) sulfate pentahydrate, 16 mg/l manganese (II) sulfate monohydrate, 10 mg/l calcium chloride dihydrate) was inoculated with the shake flask culture to an OD600 of 0.05-0.1. The cultivations were generally run at 37° C. under stirring and overpressure 0.5 bar) to control the dissolved oxygen level at 30%. The pH was controlled at pH=7 and a glucose feed was initiated 3 h after inoculation. After 17.5 h the temperature was lowered to 33° C., and after 18 h 0.6 mM of Isopropyl-β-D-thiogalactopyranoside (IPTG) was added for induction of protein production. The cultivations were stopped after 27-30 h.

Cell concentration: The cultivations were harvested either by centrifugation or tangential flow filtration. Centrifugation was performed at 9,800×g for 15 min at 23° C. and the supernatant was discarded. To reflect a large-scale separator, the cell pellet was resuspended giving about 700 g/kg cell slurry using 10 mM sodium phosphate, pH 7.0. Tangential flow filtration was run on 2×0.5 m² 1000 kDa-filters of regenerated cellulose (P2C01 MV05, Merck-Millipore), where the cultivation was concentrated to one third, followed by diafiltration with three diafiltration volumes of 50 mM sodium acetate buffer, pH 6.0.

Heat-induced extraction using a static mixer system: 10 mM sodium phosphate, 2 mM EDTA, pH 7 (expected to result in a pH of 6.5 during the heat treatment), [Heat releasing buffer 1], was heated to 91-95° C. in the media preparation tank of a multifermenter system (System Greta, Belach Bioteknik). In two separate heat treatment runs, peristaltic pumps were used to lead the 23° C. cell concentrate (−1.6 L and −4.3 L, respectively) and the heated Heat releasing buffer 1, respectively, to the static mixer (PMS3, ESSKA.se Industriteknik) with a flow rate of 30 ml/min and 137 ml/min, respectively, resulting in a 5.6 times dilution of the cell concentrate. After mixing, the cell suspension was led to the holding unit (Pumpsil® 6.4×1.6 mm tubing with a volume of 56 cm³, Watson Marlow) placed in a water bath set at 76-78° C. The resulting set-up led to a heating of the cell suspension to approximately 76° C. (operating temperature) with a holding time of 20 s. After heating and holding, the cell suspension was led to a cooling coil (S30, Bryggbolaget) placed in a bucket with ice water. Ice was added repeatedly to the water in order to keep the temperature at −25° C., in the heat treated cell suspension.

Heat-induced extraction using a fermenter: The cell concentrate was mixed with 50 mM sodium acetate buffer pH 6.0 followed by addition of EDTA to a final concentration of 2 mM and pH adjustment to pH 6.5 using 0.5 M disodium hydrogen phosphate, resulting in a 3.8 times dilution of the cell concentrate. Heating of the cell suspension was performed at 76° C. for 3 min using the heating system of the jacketed fermenter BR20 (Belach Bioteknik). The total time for heating, holding and cooling to 25° C. was approximately 1 h, simulating a large-scale heat treatment in a 200 L fermenter.

Protein analysis: Quantification was made by small-scale affinity chromatography purification of a minor fraction, followed by Abs280 measurements of the purified eluate.

### Results

Quantification of the product in the heat treated cell suspension using a static mixer system showed in average 100% recovery in two representative runs. As a comparison, using the fermenter heat treatment procedure resulted in a recovery of 87%. The resulting process improvement, in terms of recovery of product, using the static mixer thus was 15%.

### Example 2, Heat-Induced Extraction of BPEP02

The description in this Example refers to cultivation, heat-induced extraction involving use of a static mixer and subsequent analysis of an approximately 19 kDa polypeptide, referred to as BPEP02, comprising two different Z variants (Z02a and Z02b) and an albumin binding domain derived from GA3 of Streptococcal protein G. A comparison with heat treatment using a fermenter is included.

### Materials and Methods

Cultivation: The scale of the cultivations was either 2 L or 20 L. The cultivation was performed essentially as described in Example 1, with the exceptions that the final OD600 during RCB preparation was 0.80, the temperature was lowered to 31° C. after 17.5 h of cultivation.

Cell concentration: The cultivation was harvested by centrifugation or tangential flow filtration. Centrifugation was performed at 15,900×g for 25 min at 4° C. and the supernatant was discarded. Tangential flow filtration was run on 2×0.5 m² 1000 kDa-filters of regenerated cellulose (P2C01 MV05, Merck-Millipore), where the cultivation was concentrated to one third, followed by diafiltration with three diafiltration volumes of 10 mM phosphate buffer, pH 8.

Heat-induced extraction using a static mixer system: The cells were frozen before subjected to heat treatment. 25 mM sodium phosphate, 2 mM EDTA, pH 8 (expected to result in a pH of 7.3 during the heat treatment), [Heat releasing buffer 2], was heated to 91-95° C. in the media preparation tank of a multifermenter system (System Greta, Belach Bioteknik). Two peristaltic pumps were used to lead the 23° C. cell concentrate (˜6 L) and the heated Heat releasing buffer 2, respectively, to the static mixer (PMS3, ESSKA.se Industriteknik) with a flow rate of 25 ml/min and 142 ml/min, respectively resulting in a 6.7 times dilution. After mixing, the cell suspension was led to the holding unit (S30, Bryggbolaget, with an estimated volume of 500 cm³) placed in a water bath set at 76° C. The resulting set-up led to a heating of the cell suspension to approximately 76° C. (operating temperature) for 3 min. After heating, the cell suspension was led to a cooling coil (S30, Bryggbolaget) placed in a bucket with ice water. Ice was added repeatedly in order to keep the final temperature at ˜25 ° C., in the heat treated cell suspension.

Heat release using a fermenter: The cells were frozen before they were subjected to heat treatment. The cell concentrate was mixed with 179 mM phosphate 11 mM citrate buffer followed by addition of EDTA to a final concentration of 2 mM, resulting in a 5 times dilution of the cells. Resulting pH of the cell suspension was 7.3. Heating of the cell suspension was performed at 76° C. for 3 min using the heating system of the fermenter BR20 (Belach Bioteknik). The total time for heating, holding and cooling to 25° C. was 75 min.

Protein analysis: Quantification was made by small-scale affinity chromatography purification of a minor fraction, followed by Abs280 measurements of the purified eluate.

### Results

Quantification of the product in the heat treated cell suspension using a static mixer system showed 69% recovery. As a comparison, using the fermenter heat treatment procedure, the recovery was 46%. The resulting process improvement, in terms of recovery of product, using the static mixer thus was 50%.

### Example 3, Heat-Induced Extraction of BPEP03

The description in this Example refers to cultivation, heat-induced extraction involving use of a static mixer, and subsequent analysis of an approximately 19 kDa polypeptide, referred to as BPEP03, comprising two copies of a Z variant (Z03) and an albumin binding domain derived from GA3 of Streptococcal protein G. A comparison with heat treatment using a fermenter is included.

### Materials and Methods

Cultivation: The scale of the cultivation was 1 L. The cultivation was performed essentially as described in Example 1, with the exceptions that the temperature was lowered to 31° C. after 17.5 h hours of cultivation.

Cell concentration: The cultivation was harvested by centrifugation. Centrifugation was performed at 9,800×g for 15 min, 23° C. and the supernatant was discarded. To reflect a large-scale separator, the cell pellet was resuspended in a 10 mM phosphate buffer, pH 7.4, yielding a −700 g/kg cell slurry.

Heat-induced extraction using a static mixer system: 25 mM phosphate, 2 mM EDTA, pH 8.5, [Heat releasing buffer 3], was heated to 91-95° C. in the media preparation tank of a multifermenter system (System Greta, Belach Bioteknik). Two peristaltic pumps were used to lead the 23° C. cell concentrate (˜0.15 L) and the heated Heat releasing buffer 3, respectively, to the static mixer (PMS3, ESSKA.se Industriteknik) with a flow rate of 25 ml/min and 114 ml/min, respectively resulting in a 5.6 times dilution of the cell concentrate. After mixing, the cell suspension was led to the holding unit (S30, Matrevolution, estimated to a holding volume of 417 ml) placed in a water bath set at 77.6° C. The resulting set-up led to an instantaneous heating of the cell suspension to 75° C., pH ˜7.4, with a 3 min holding time. After heating, and holding the suspension at the operating temperature, the cell suspension was led to a cooling coil (S30, Matrevolution) placed in a bucket with ice water. Ice was added repeatedly in order to keep the final temperature at ˜25° C., in the heat treated cell suspension.

Heat-induced extraction using a fermenter: The cell concentrate was mixed with 25 mM phosphate, 2 mM EDTA, pH 8.5 giving the same proportions of cell concentrate and buffer as described for the static mixer procedure in the section above. Heating of the cell suspension was performed to simulate a large-scale heat treatment in a >200 L fermenter using the heating system of the fermenter BR20 (Belach Bioteknik). Thus, a heating profile was set in the fermenter, where heating from 25° C. to 75° C. was set to take approximately 50 min followed by a holding step at 75° C. for 3 min and finally cooling to 25° C. in 30 min. The total time for heating, holding and cooling was approximately 83 min.

Protein analysis: Quantification was made by small-scale affinity chromatography purification of a minor fraction, followed by Abs280 measurements of the purified eluate. Furthermore, SDS-PAGE analysis of the purified fraction was performed to assess product related impurities, such as dimerization and degradation.

### Results

Quantification of the product in the heat treated cell suspension from heat treatment with a static mixer showed 71% recovery, whereas heat treatment in a fermenter resulted in a recovery of 33%. Thus, the resulting process improvement, in terms of recovery of product, using the static mixer was 115%.

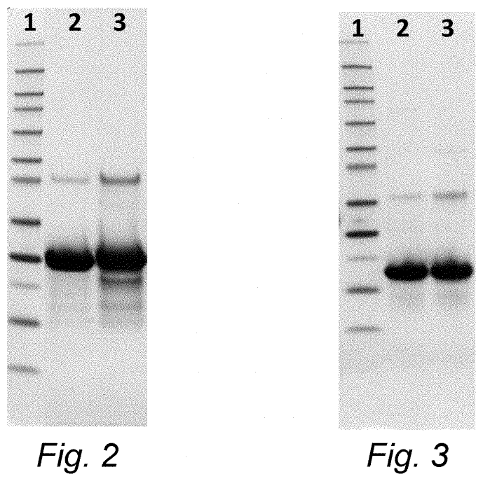

During the comparison also a major advantage in terms of product quality was detected for the static mixer heat treated sample compared to fermenter heat treated as depicted in the SDS-PAGE analysis. FIG. 2 shows the SDS-PAGE analysis of affinity purified lysates containing BPEP03 after heat treatment with static mixer (Lane 2) and heat treatment in fermenter (Lane 3), respectively, loaded on the gel at 8 μg. Novex® Sharp Protein Standard (Mw: 260, 160, 110, 80, 60, 50, 40, 30, 20, 15, 10, 3.5 kDa) was loaded in Lane 1. The heat treatment sample from fermenter shows both more degradation and dimerization. Besides the increased recovery and favourable sample profile when using a static mixer, the process of using the static mixer for heat treatment enables industrial production, not feasible with a fermenter based heat treatment.

### Example 4, Heat-Induced Extraction of BPEP04

The description in this Example refers to cultivation, heat-induced extraction involving use of a static mixer, and subsequent analysis of an approximately 14 kDa albumin binding protein referred to as BPEP04 comprising the two albumin binding domains GA2 and GA3 of Streptococcal protein G, and a C-terminal cysteine residue. A comparison with heat treatment using a fermenter is included.

### Materials and Methods

Cultivation (1 L scale), heat induced extraction using the lab-scale static mixer system and a fermenter, respectively, as well as protein analysis were performed essentially as described in Example 3.

### Results

Quantification of the product in the heat treated cell suspension from heat treatment with static mixer or heat treatment with fermenter, both showed 100% recovery. However, the comparison showed an advantage in terms of product quality for the static mixer heat treated sample compared to fermenter heat treated as depicted in the SDS-PAGE analysis. FIG. 3 shows the SDS-PAGE analysis of affinity purified lysates containing BPEP04 after heat treatment with static mixer (Lane 2) and heat treatment in fermenter (Lane 3), respectively, loaded on the gel at 8 μg. Novex® Sharp Protein Standard was loaded in Lane 1. The heat treatment sample from fermenter shows both more degradation and higher fractions of multimeric forms (dimers, trimers, and tetramers).

### Example 5, Heat-Induced Extraction of BPEP05

The description in this Example refers to cultivation, heat-induced extraction involving use of a static mixer, and subsequent analysis of an approximately 6.7 kDa polypeptide, referred to as BPEP05, comprising one copy of a Z variant (Z04) and a C-terminal cysteine residue. A comparison with heat treatment using a fermenter is included.

### Materials and Methods

Cultivation (1 L scale) and heat induced extraction using the static mixer system and a fermenter, respectively, were performed essentially as described in Example 3, with the exception that instead of establishing an RCB, the shake flask starter culture was inoculated with a culture run with TSB+YE-medium, which had been incubated for 5 h at 30° C. Quantification of the product was carried out by ultra-performance liquid chromatography-mass spectrometry (UPLC-MS).

### Results

UPLC-MS quantification of the product in the heat treated cell suspension from heat treatment with static mixer and fermenter, respectively, showed 43% better recovery when using static mixer compared to heat treatment in fermenter.

### Example 6, Large-Scale Heat Release of BPEP01

Production of BPEP01 using a heat treatment according to the present invention in a large scale process has successfully been demonstrated. Cultivation and harvest were performed essentially as described in Example 1, but in a 100 L cultivation scale and using a disc stack centrifuge (GEA Westfalia) for cell concentration. Heat treatment was performed in a static mixer with the same proportions of cell suspension and [Heat releasing buffer 1] as in Example 1 and using a heat treatment system denoted S175, with a holding unit volume of 13.6 L and a total flow rate of 6.8 L/min, giving 2 min hold time at an operating temperature of 76±1° C. The recovery from the large-scale run was 100% and corresponds well to the recovery obtained in small scale run, described in Example 1. Thus, the result of this experiment confirms scalability and industrial applicability.

### Example 7, Large-Scale Heat Release of BPEP02

Three batches, of which two were performed under GMP (Good Manufacturing Practice), have successfully been run using a heat treatment according to the present invention in a large-scale process, for the production of BPEP02. Cultivation and harvest were performed essentially as described in Example 2, in a 300 L cultivation scale. Heat treatment was performed in a static mixer with the same proportions of cell suspension and [Heat releasing buffer 2] as in Example 2 and using a heat treatment system denoted S163, with a holding unit volume of 26 L and a total flow rate of 8.67 L/min, giving 3 min hold time at an operating temperature of 80-84° C. The recoveries from the three large-scale runs were 73-85% and correspond well to the recovery obtained in small scale run described in Example 2. Thus, the result of this experiment confirms scalability and industrial applicability.

1. A method of extracting a cytoplasmic or periplasmic protein, the method comprising:
providing of a first cell suspension comprising cells, the cells containing a cytoplasmic or periplasmic protein of interest to be extracted, the first cell suspension having a first temperature, and
heating of the first cell suspension to an operating temperature, wherein at least a fraction of the cells is subject to heat-induced lysis and at least a fraction of the cytoplasmic or periplasmic protein of interest to be extracted is not subject to irreversible denaturation, and wherein the heating of the first cell suspension comprises:
providing an aqueous solution, the aqueous solution having a second temperature that is higher than the first temperature, and
mixing of the first cell suspension with the aqueous solution, thereby obtaining a second cell suspension, the second cell suspension having a third temperature that is higher than the first temperature.)

2. The method according to claim 1, wherein the heating of the first cell suspension further comprises:
heating of the second cell suspension from the third temperature to the operating temperature.

3. The method according to claim 1, wherein the third temperature is no more than 10° C. lower than the operating temperature.

4. The method according to claim 1, wherein the third temperature is the operating temperature.

5. The method according to claim 1, wherein the method further comprises:
maintaining of the second cell suspension at the operating temperature for a time period of about 1 s to 20 min, about
10 s to 20 min, about 1 s to 10 min, about 10 s to 10 min, about 1 s to 5 min, about 10 s to 5 min, about 10 s to 4 min, about 10 s to 30 or about 1 min to 4 min.

6. The method according to claim 1, wherein the method further comprises:
cooling of the second cell suspension from the operating temperature to a fourth temperature, wherein the fourth temperature is a temperature at which at least a fraction of reversibly denatured cytoplasmic or periplasmic protein of interest to be extracted is subject to renaturation.

7. The method according to claim 1, wherein the method further comprises:
separating the cytoplasmic or periplasmic protein of interest from cell debris and/or native host cell proteins.

8. The method according to claim 1, wherein
the first temperature is about in the range of 0 to 37° C., preferably in the range about 2 to 37° C., more preferably in the range about 8 to 30° C., more preferably in the range or about 18 to 25° C.

9. The method according to claim 1, wherein the fourth temperature is about 2 to 37° C., about 8 to 30° C., about 25 to 37° C., or about 18 to 25° C.

10. The method according to claim 6, wherein the time at a temperature that is higher than both the first temperature and the fourth temperature is no more than 20 min, about 1 s to 20 min, about 10 s to 20 min, about 10 min, about 1 s to 10 min, about 10 s to 10 min, about 5 min, about 1 s to 5 min, about 10 s to 5 min.

11. The method according to claim 1, wherein the cells are prokaryotic cells or eukaryotic cells.

12. The method according to claim 1, wherein the cytoplasmic or periplasmic protein of interest to be extracted comprises a three-helix bundle protein domain of a bacterial receptor protein, or a variant thereof.

13. The method according to claim 1, wherein the mixing of the first cell suspension with the aqueous solution is performed in a static mixer or in an agitated vessel.

14. A system for extracting a cytoplasmic or periplasmic protein, the system comprising:
a cell suspension supply conduit having an inlet and an outlet, the inlet of the cell suspension supply conduit being connectable to a cell suspension container;
an aqueous solution supply conduit having an inlet and an outlet, the inlet of the aqueous solution supply conduit being connectable to an aqueous solution container;
a static mixer having at least one inlet and an outlet, the at least one inlet of the static mixer being in liquid communication with the outlet of the cell suspension supply conduit and with the outlet of the aqueous solution supply conduit;
a first heat exchanger having an inlet for cell suspension to be heated and an outlet for heated cell suspension, the inlet of the first heat exchanger being in liquid communication with the outlet of the static mixer;
a second heat exchanger having an inlet for cell suspension to be cooled and an outlet for cooled cell suspension, the inlet of the second heat exchanger being in liquid communication with the outlet of the first heat exchanger;
a discharge conduit having an inlet and an outlet, the inlet of the discharge conduit being in liquid communication with the outlet of the second heat exchanger and the outlet of the discharge conduit being connectable to a protein suspension container or to a protein suspension treatment system.

15. The system of claim 14, further comprising a holding conduit providing liquid communication between the outlet of the first heat exchanger and the inlet of the second heat exchanger, the holding conduit being surrounded by a jacket, a vessel, a heat insulating material, a heating blanket, or an electrical heating blanket.

16. The method according claim 1, wherein the third temperature is no more than 5° C. lower than the operating temperature.

17. The method of claim 1, wherein the operating temperature is below 90° C., about 20 to 90° C., about 40 to 90° C., about 50 to 90° C., about 60 to 90° C., about 70 to 90° C., about of 70 to 85° C., or about 75 to 85° C.

18. The method of claim 1, wherein the second temperature is below 110° C., about 40 to 110° C., about 50 to 110° C., about 60 to 99° C., about 70 to 99° C., about 80 to 99° C., about 90 to 99° C., about 90 to 95° C.

19. The method according to claim 1, wherein the third temperature is below 90° C., about 40 to 90° C., about 50 to 90° C., about 60 to 85° C. about 65 to 85° C., about 65 to 80° C., about 65 to 78° C., or about 68 to 78° C.

20. The method according to claim 11, wherein the prokaryotic cells are E. coli.
